(19) United States
(12) Patent Application Publication
(10) Pub. No.: US 2016/0340264 A1
(43) Pub. Date: Nov. 24, 2016

(54) MICROALGAE AS A MINERAL VEHICLE IN AQUAFEEDS

(71) Applicant: Heliae Development LLC, Gilbert, AZ (US)

(72) Inventor: Eneko GANUZA, Phoenix, AZ (US)

(21) Appl. No.: 15/221,263

(22) Filed: Jul. 27, 2016

Related U.S. Application Data
(63) Continuation of application No. 13/766,306, filed on Feb. 13, 2013, now abandoned.
(60) Provisional application No. 61/598,235, filed on Feb. 13, 2012.
(60) Provisional application No. 61/601,970, filed on Feb. 22, 2012.

(51) Int. Cl. C05D 9/02; A23K 10/22; A23K 20/158; A23K 20/22; A23K 20/24; A23K 20/26; A23K 20/20; A23K 50/10; A23K 50/20; A23K 50/30; A23K 50/40; A23K 50/75; A23K 50/80; C05B 17/00; C12P 1/00; C12N 13/00; C12N 1/12; A23K 10/12

(52) U.S. Cl. CPC C05D 9/02; A23K 10/12; A23K 10/22; A23K 20/158; A23K 20/22; A23K 20/24; A23K 20/26; A23K 20/30; A23K 50/10; A23K 50/20; A23K 50/30; A23K 50/40; A23K 50/75; A23K 50/80; C05B 17/00; C12P 1/00; C12N 13/00; C12N 1/12

(57) ABSTRACT

Disclosed herein are aquafeed, animal feed and fertilizer compositions comprising microalgae enriched with minerals and a method of enriching microalgae with minerals in non-metabolized form. Specifically, the method includes the creation of an enriched microalgae product through the assimilation, reversible chelation, and absorption of supplemental minerals required in the diet of adult fish and other aquatic animals which minimizes leaching of the supplemental minerals before ingestion by the fish. Additionally, the enriched microalgae product can be used as both a direct feed or fertilizer, or as part of an aquafeed, non-aquatic animal feed, or plant fertilizer mixture. The combination and proportion of the minerals can be adjusted to the animal or plant receiving the mineral enriched algae composition.

### CROSS-REFERENCES TO RELATED APPLICATIONS

This application claims the benefit of U.S. Provisional Patent Application No. 61/598,235, filed Feb. 13, 2012 and U.S. Provisional Patent Application No. 61/601,970, filed Feb. 22, 2012, and incorporates the disclosure of each application by reference. To the extent that the present disclosure conflicts with any referenced application, however, the present disclosure is to be given priority.

### BACKGROUND

Aquaculture is the fastest growing animal-food-producing sector, with an average annual growth rate of 6.6% from 1970 to 2008 in per capita supply of food fish from aquaculture for human consumption according to the Food and Agriculture Organization of the United Nations (FAO) statistics. Due to this growth, the FAO reports that aquaculture now accounts for approximately 46% of the total food fish supply, which equates to over 50 million tons of fish. Decreasing the dependence of the aquafeed industry on fisheries may help aquaculture sustain this level of growth. Specifically, vegetable sources such as soybean, linseed, canola, etc., have been proposed to replace a portion of the fish oil and fishmeal currently used in fish feeding formulations.

Fish diets may comprise a combination of proteins, lipids, amino acids, vitamins, and trace minerals. Trace minerals for fish may comprise elements such as: Arsenic, Chromium, Cobalt, Copper, Fluorine, Iron, Iodine, Lead, Lithium, Manganese, Molybdenum, Nickel, Selenium, Silicon, Vanadium, and Zinc. The ranges for trace minerals specific for fish diets may include (in mg mineral per kg dry diet): 30-170 mg/kg Iron, 1-5 mg/kg Copper, 2-20 mg/kg Manganese, 15-40 mg/kg Zinc, 0.05-1.0 mg/kg Cobalt, 0.15-0.5 mg/kg Selenium, and 1-4 mg/kg Iodine. Although fish may uptake some amounts of these minerals from the water through their gills, receiving the minerals in their diet via the digestive system may be a more efficient method of mineral delivery.

Vegetable sources may provide many of the essential lipids and amino acids present in fish meal, however one drawback with vegetable sources has been mineral deficiencies. The replacement of fish meal by vegetable sources requires an extra supplementation of minerals such as Selenium, Manganese, Zinc, Iron, Copper and Chromium three complexes. Minerals may be supplemented in an aquafeed diet as water-soluble inorganic salts, but the disadvantage of this method may be the leaching of a large portion of the minerals before being ingested by the fish. The loss of minerals in the water before ingestion by the fish may result in costs associated with wasted minerals and inefficient delivery of nutrients to the fish.

A more efficient delivery of minerals to fish may occur when the minerals are in a bioavailable state. The bioavailability of the trace minerals may be subject to a number of factors, including: the concentration of the nutrient, the form of the nutrient, the particle size of the diet, the digestibility of the diet, the nutrient interactions which may be either synergistic or antagonistic, the physiological and pathological conditions of the fish, waterborne mineral concentration, and/or the species under consideration. In an effort to ensure sufficient bioavailability of the required amount of minerals, fish feeds may be enriched with trace elements at higher concentration than needed by the fish due to the limited information on leaching and bioavailability.

Adding trace elements at higher than needed concentrations may introduce a number of potential complications. One such potential complication may be that the high concentration of minerals has the potential to interact with fatty acid oxidative processes in the fish diet through the formation of hydroperoxides. In addition, minerals leaching from the aquafeed may have the potential to negatively impact the environment. The excessive leaching of minerals may stimulate phytoplankton production and increase oxygen demand. Leached minerals may also have the potential to stimulate the development of macroalgal beds and influence the benthonic ecosystem. Accordingly, a plurality of unintended consequences may be produced by leaching and high concentrations of minerals may harm the fish and aquatic environment.

### SUMMARY

Disclosed herein are aquafeed, animal feed and fertilizer compositions comprising microalgae enriched with minerals and a method of enriching microalgae with minerals in non-metabolized form. Specifically, the method includes the creation of an enriched microalgae product through the assimilation, reversible chelation, and absorption of supplemental minerals required in the diet of adult fish and other aquatic animals which minimizes leaching of the supplemental minerals before ingestion by the fish. Additionally, the enriched microalgae product can be used as both a direct feed or fertilizer, or as part of an aquafeed, non-aquatic animal feed, of plant fertilizer mixture. The combination and proportion of the minerals can be adjusted to the animal or plant receiving the mineral enriched algae composition.

### DETAILED DESCRIPTION

The present invention may be described in terms of functional block components and various processing steps. Such functional blocks may be realized by any number of components configured to perform the specified functions and achieve the various results. For example, the present invention may employ various process steps, apparatus, systems, methods, etc. In addition, the present invention may be practiced in conjunction with any number of systems and methods for providing microalgae as a vegetable source for aquafeed, and the system described is merely one exemplary application for the invention. Various representative implementations of the present invention may be applied to any type of live aquaculture. Certain representative implementations may include, for example, providing the microalgae preparation to the aquaculture to at least partially meet the nutritional needs of the aquaculture.

The particular implementations described are illustrative of the invention and its best mode and are not intended to otherwise limit the scope of the present invention in any way. For the sake of brevity, conventional manufacturing, connection, preparation, and other functional aspects of the system may not be described in detail. Many alternative or additional functional relationships or physical connections may be present in a practical system.

Various embodiments of the invention may provide methods, apparatus, and systems for providing an aquafeed vegetable source comprising microalgae that may be grown quickly and/or year round to ensure a readily available supply. In some embodiments, microalgae may provide an alternative vegetable source for aquafeed which may possess a beneficial amino acid profile and/or a high unsaturated fatty acid profile. Microalgae may also provide a bio-absorption capacity. For example, microalgae may absorb, chelate and/or assimilate trace minerals from a medium, even at very low concentrations. The ability to absorb, chelate, and assimilate minerals from a medium may be due to several characteristics of microalgae such as, but not limited to, a large surface to volume ratio, the presence of high-affinity metal binding groups on the microalgae cell surface, and/or efficient metal uptake and storage systems. These mineral uptake characteristics of microalgae may provide a potential advantage over other vegetable sources for fish feed.

Microalgae, such as Nannochloropsis, Chlorella or Scenedesmus, may be a rich source of minerals, fatty acids of chain length C10-C24, and proteins, which may provide a nutritious and natural source for feeding fish. For example, every 100 g of Nannochloropsis contains 972 mg Calcium (Ca), 533 mg Potassium (K), 659 mg Sodium (Na), 316 mg Magnesium (Mg), 103 mg Zinc (Zn), 136 mg iron (Fe), 3.4 mg Manganese 35.0 mg Copper (Cu), 0.22 mg Nickel (Ni), and >0.1 mg Cobalt (Co). Additionally, Nannochloropsis may possess an essential fatty acid and amino acid profile having nutritional value. Together, the growth characteristics and nutritional composition may make microalgae a leading vegetable source alternative for aquafeed.

In addition to the growth characteristics and nutritional composition, microalgae may have other characteristics associated with their nanoparticle properties that provide unique benefits as an aquafeed over other vegetable sources. The large surface to volume ratio and the presence of high affinity, metal binding groups confers the microalgae the ability to adsorb trace minerals from a medium. The microalgae cells may sequestrate soluble ions from water and concentrate them at their specific requirements. For example, Chlorella and Scenedesmus may absorb Zn2+ and Cr6+ and concentrate them above 0.2% of dry weight. This ability of microalgae to bind metals and minerals may be subject to multiple variables such as, but not limited to, the pH of the water medium, the temperature of the water medium, the concentration of the minerals, the mass of the microalgae, and the time allowed for the metal to hind to the surface of the microalgae. In some embodiments, these variables may be adjusted as parameters in a method of making an aquafeed to produce an aquafeed of a desired composition, for a desired purpose, or at a desired cost.

Also, in some embodiments, the microalgae cells may be able to prevent toxicity at high concentrations by preventing the indiscriminate entry of the minerals into the microalgae cell. Minerals may reversibly chelate to the microalgae cell wall or the extra-cellular polymers before they interact with the cellular metabolism. Mineral chelation may refer to a mineral that is bound to amino acids or proteins. Mineral chelation may comprise the metabolization of the mineral by the microalgae into its organic configuration. Unlike the reversible chelation that occurs in the cell wall of the microalgae, the chelated minerals will remain bound to the organic molecule during pH change conditions, such as digestion.

This chelating process provides more stability to metal ions and reduces the ability of the ions to leach or form soluble precipitates. Following the trace metal absorption, the microalgae cell can uptake the minerals and form peptide complexes, commercially known as “chelated minerals”, that may increase the tolerance to high ion concentrations. Alternatively, the microalgae cell can reverse the chelation reaction and release the minerals, for example when the pH of the suspension decreases. The reversible properties of the mineral chelation in microalgae provide a clear benefit to its application to the aquafeed industry, for instance the minerals can be released into the fish digestive track in response to a change in the pH. The acid digestion of the fish will release the minerals chelated to the microalgae cell wall, therefore avoiding any unwanted leaching of the essential nutrients in the water before digestion by the fish. Therefore, the minerals will be delivered in the appropriate place and timing to maximize the efficiency of the fish feeding process.

The minerals may be absorbed, reversibly chelated or assimilated by the microalgae through a variety of mechanisms. Examples of such mechanisms comprise two active absorbing substances in a Chlorella cell wall: the cellulose microfibrils and the sporopollenin. Additionally, the mucopolysaccharides covering the cell wall possesses a similar mechanism to the ion exchange resigns that are used to reversibly Chelate heavy metals in industrial wastewater treatment. Also, the microalgae can uptake and assimilate the minerals in their organic forums known as “chelated minerals”, which further enhances the digestibility of the minerals by the fish, as opposed to the inorganic form of the minerals most commonly used in aquafeeds. By binding the supplemental minerals through chelating, assimilating and absorbing, the enriched microalgae are acting, as a carrier or vehicle for supplying the minerals to adult fish, which is distinguishable from adding trace minerals to a culture of microalgae for the microalgae to metabolize. The metabolized minerals provide nutrition to the microalgae cell for growth, whereas the bound minerals provide nutrition directly to the adult fish.

The supplemental minerals may comprise any suitable mineral that may provide nutrition to the microalgae cell and/or an aquatic animal and may come from a variety of sources, including purchased concentrations of the minerals. For example, the supplemental minerals may comprise various sources of boron, bromine, calcium, chloride, chromium, cobalt, copper, fluorine, iodine, iron, lithium, magnesium, manganese, molybdenum, nickel, phosphorus, potassium, selenium, sodium, silicon, sulphur, vanadium, and/or zinc. The calcium sources may comprise: calcium carbonate (CaCO₃); monocalcium phosphate, monohydrate (Ca(H₂PO₄)₂.H₂O); dicalcium phosphate, anhydrous (CaHPO₄); dicalcium phosphate, dihydrate (Ca(HPO₄.2H₂O); tricalcium phosphate (Ca₃(PO₄)₂), calcium sulphate (CaSO₄); bonemeal; oystershell grit; and ground limestone (CaCO₃). The chloride sources may comprise: sodium chloride (NaCl) and potassium chloride (KCl). The chromium sources may comprise: chromium (III) chloride (CrCl₃); chromium (III) chloride, hexahydrate (CrCl₃.6H₂O); and chromium picolinate (Cr(C₆H₄NO₂)₃). The cobalt sources may comprise: cobalt chloride, pentahydrate (CoCl₂.5H₂O); cobalt chloride, hexahydrate (CoCl₂.6H₂O); and cobalt sulphate, monohydrate (CoSO₄.H₂O). The copper sources may comprise: copper sulphate (CuSO₄); copper sulphate, pentahydrate (CuSO₄.5H₂O); copper chloride (CuCl₂); copper (II) oxide (CuO); and copper (I) hydroxide (Cu(OH)₂). The iodine sources may comprise: potassium iodide (KI); potassium iodate (KIO₃); calcium iodate (Ca(IO₃)₂); sodium iodide (NaI); and ethylenediamine dihydriodide (C₂H₈N₂.2HI). The iron sources may comprise: ferrous sulphate, heptahydrate (FeSO₄.7H₂O); ferrous (II) carbonate (FeCO₃); and ferrous oxide (FeO). The magnesium sources may comprise: magnesium chloride (MgCl₂.6H₇O); magnesium oxide (MgO); magnesium carbonate (MgCO₃); dimagnesium phosphate, trihydrate (MgHPO₄.3H₂O); magnesium sulphate (MgSO₄) and magnesium sulphate, heptahydrate (MgSO₄.7H₂O). The manganese sources may comprise: manganese oxide (MnO); manganese dioxide (MnO); manganese carbonate (MnCO₃); manganese chloride, tetrahydrate (MnCl₂4H₂O); manganese sulphate (MnSO₄); manganese sulphate, hydrate (MnSO₄.H₂O); and manganese sulphate, tetrahydrate (MnSO₄.4H₂O). The molybdenum sources may comprise: sodium molybdate, dihydrate (Na₂MoO₄.2H₂O) and sodium molybdate, pentahydrate (NaMO₄.5H₂O). The phosphorus sources may comprise: monocalcium phosphate, monohydrate (Ca(H₂PO₄)₂.H₂O); dicalcium phosphate, anhydrous (CaHPO₄); dicalcium phosphate, dihydrate (CaHPO₄.2H₂O); tricalcium phosphate (Ca₃(PO₄)₂); potassium orthophosphate (K₂HPO₄); potassium dihydrogen orthophosphate (KH₂PO₄); sodium hydrogen orthophosphate (Na₂HPO₄); sodium dihydrogen orthophosphate, hydrate (NaH₃PO₄.H₂O);, sodium dihydrogen orthosphosphate, dihydrate (NaH₃PO₄.2H₂O); dimagnesium phosphate, trihydrate (MgHPO₄.3H₂O); and rock phosphate ((Ca₃(PO₄)₂)₃CaF₂). The potassium sources may comprise: potassium chloride (KCL); potassium carbonate (K₂CO₃); potassium bicarbonate (KHCO₃); potassium acetate (KC₂H₃O₂); potassium orthophosphate (K₃PO₄); and potassium sulphate (KSO₄). The selenium sources may comprise: sodium selenite (Na₂SeO₃) and sodium selenate (NaSeO₄). The sodium sources may comprise: sodium chloride (NaCl); sodium bicarbonate (NaHCO₃); and sodium sulphate (Na₂SO₄). The zinc sources may comprise: zinc carbonate (ZncO₃); zinc chloride (ZnCl₂); zinc oxide (ZnO); zinc sulphate (ZaSO₄); zinc sulphate, hydrate (ZnSO₄.H₂O); and zinc sulphate heptahydrate (ZnSO₄.7H₂O). In some embodiments, the supplemental minerals are added to de-ionized water and then administered to the microalgae.

Electrodes receiving electrical current in direct, alternating, pulsed or any other form from a power source are known to degrade over time and leach electrode material. Electrodes that are submerged in an aqueous medium will leach the electrode material into the aqueous medium. Applying an electric field to an aqueous culture of microalgae through electrodes submerged in the aqueous culture is also known in the art to cause flocculation among the microalgae by mechanisms such as, but not limited to, changing the surface charge of the microalgae cells to reduce electrostatic repulsion, and the leached electrode material acting as a flocculent or flocculating aid. In some embodiments, electrodes comprised of an electrode material of a desired mineral composition as described above, such as but not limited to copper, zinc, iron, and alloys thereof, are submerged in an aqueous culture comprising microalgae. When current is applied to the electrodes, the electrode material degrades and leaches into the aqueous medium which supplies the supplemental minerals for uptake by the microalgae. The microalgae assimilate, reversibly chelate, and absorb the leached electrode material to produce a microalgae product enriched with the desired mineral composition in a non-metabolized form. In further embodiments, the application of an electric field by the electrodes simultaneously causes flocculation of the microalgae which results in a flocculated mass of mineral enriched microalgae.

In an exemplary embodiment of the present invention, a method of making the microalgae product enriched with non-metabolized minerals may comprise growing a culture of microalgae in a culturing vessel containing an aqueous culture medium and at least one pair electrodes submerged in the aqueous culture medium. The at least one pair of electrodes may comprise an electrode material comprising a mineral specific to a nutritional profile of an animal. The electric current may be applied to the at least one pa of electrodes sufficient to cause the electrode material to leach into the aqueous culture medium. The microalgae may be incubated to facilitate the microalgae assimilating, reversibly chelating, and/or absorbing the electrode material to produce a microalgae product enriched with non-metabolized minerals specific to the profile of nutritional requirements of the animal. The microalgae product enriched with non-metabolized minerals may be harvested to separate the microalgae product from the aqueous culture medium.

The enriched microalgae may be administered to the fish in various forms. In sonic embodiments, the enriched microalgae comprise a suspension microalgae in water. In some embodiments, the enriched microalgae comprise a paste or cake resulting from dewatering the microalgae culture to a desired percent of solids. In some embodiments, the enriched microalgae comprises a dried free flowing powder or flakes. for use as an ingredient in the dietary mixing and pelletizing.

A method for making a microalgae product enriched with non-metabolized minerals. comprises the steps of: growing a culture of microalgae in an aqueous culture medium; harvesting the microalgae by separating the microalgae from the aqueous culture medium; adding supplemental minerals specific to a profile of nutritional requirements for an animal to the microalgae; incubating the microalgae and the supplemental minerals to facilitate the microalgae assimilating, reversibly chelating, and absorbing the supplemental minerals to produce a microalgae product enriched with non-metabolized minerals specific to the profile of nutritional requirements of the animal. In one embodiment, the method may further comprise dewatering the microalgae product enriched with minerals to further reduce the water content of the microalgae product. In another embodiment, the method may further comprise stabilizing the microalgae product enriched With minerals. Additionally various embodiments of the present invention, the supplemental minerals may be added to the microalgae before or after the step of harvesting the microalgae.

In some embodiments of the present invention, the mineral supplement composition for an aquatic animal may comprise the microalgae product enriched with at least one mineral from the group consisting of arsenic, chromium, cobalt, copper, fluorine, iron, iodine, lead, lithium, manganese, molybdenum, nickel, selenium, silicon, vanadium, zinc in a non-metabolized form.

In another embodiment of the present invention, the mineral supplement composition for a non-aquatic animal may comprise a microalgae product enriched with at least one mineral from the group consisting of boron, bromine, calcium, chloride, chromium, cobalt, copper, iodine, iron, magnesium, manganese, molybdenum, nickel, phosphorus, potassium, selenium, sodium, sulphur, vanadium and zinc in a non-metabolized form.

In various embodiments of the present invention, the microalgae product may comprise any suitable species of algae and/or microalgae for providing nutrition to an animal. For example, the microalgae product may comprise microalgae that are members of one of the following divisions: Chlorophyta, Cyanophyta (Cyanobacteria), and Heterokontophyta. In some embodiments, the microalgae product may comprise microalgae of the following classes: Bacillaniophyceae, Eustigmatophyceae, and Cluysophyreae. In certain embodiments, the microalgae product may comprise microalgae that are members of one of the following genera: Nannochloropsis, Chlorella, Dunaliella, Scenedesmus, Selenastrum, Oscillatoria, Phormidium, Spirulina, Amphora, and Ochromonas.

In various embodiments of the present invention, the microalgae product may comprise saltwater algal cells such as, but not limited to, marine and brackish algal species. Non-limiting examples of saltwater algal species include Nannochloropsis species and Dunaliella species. Saltwater algal cells may be found in nature in bodies of water such as, but not limited to, seas, oceans, and estuaries. Further, in some embodiments, the microalgae product may comprise freshwater microalgal cells such as, but not limited to Scenedesmus species and Haematococcus species. Freshwater microalgal cells may be found in nature in bodies of water such as, but not limited to, lakes and ponds.

In various embodiments of the present invention the microalgae product may comprise one or more microalgae species such as, but not limited to: Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureviridis, Chlorella Candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorella kessleri, Chlorella laborphora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharaphila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebuoxioides, Chlorella vulgaris, Dunaliella infusianum, Dunaliella sp., Chlorogonium, Chroomonas sp., Chrysosphaera sp., Cricosphaera sp., Crypthecodinium sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuto, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Gleocapsa sp., Gloethamnion sp., Haemotococcus pluvialis, Hymenomonas sp., Isochrysis aff. galbana, Isochrysis galbana, Lepocinclis, Micractinium, Monoraphidium minutum, Monoraphidium sp., Nannochloris sp., Nannochloropsis salino, Nannochloropsis sp., Navicula acceptata, Navicula biskanterae, Navicula pseudotenelloides, Navicula pelliculosa, Navicula saprophila, Navicula sp., Nephrocloris sp., Nephroselmis sp., Nitzschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis, Prototheca moriformis, Prototheca zopfii, Pseurochlorella aquatica, Pyraminimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

In various embodiments, the microalgae may be grown in any type of culturing vessel such as, but not limited to, a pond, a raceway pond, a trough, a V-trough, a tank, or a photobioreactor able to contain an aqueous medium. In some embodiments, the microalgae may be grown phototrophically. In some embodiments, the microalgae may be grown mixotrophically. In some embodiments, the microalgae may be grown heterotrophically.

During the harvesting step, the microalgae may be separated from the aqueous culture medium. The microalgae may be harvested using any method known in the art such as, but not limited to, separation by an adsorptive bubble separation device, centrifuge, dissolved air flotation (DAF), and settling. During the dewatering step, the harvested microalgae have additional water removed to decrease the water content and increase the solids content of the microalgae product. In some embodiments, dewatering may comprise the removal of at least some water from the microalgae. The microalgae may be dewatered using, any method known in the art such as, but not limited to, electrodewatering, filtration, centrifugation, adsorptive bubble separation, and pressing. In some methods microalgae, harvested or dewatered microalgae product may be dried by methods known in the art.

In some embodiments, the supplemental minerals added comprise at least one from the group comprising: calcium, chloride, chromium, cobalt, copper, iodine, iron, magnesium, molybdenum, phosphorus, potassium, selenium, sodium, and zinc. In some embodiments, the supplemental minerals are added to the microalgae within the culturing vessel, before the microalgae are harvested. In further embodiments, the supplemental minerals are added to the culturing vessel at a specific time when the microalgae are in a specified condition or state such as, but not limited to, growth phase, a period of environmental stress, and oil phase. In some embodiments, the supplemental minerals are added to the microalgae culture after the microalgae have been harvested. In further embodiments, the supplemental minerals are added to the harvested microalgae culture at a specific time duration after the microalgae have been harvested. In some embodiments, the supplemental minerals are added to the microalgae and incubated for a determined period of time, at a determined temperature, and at a determined pH.

The timing at which the supplemental minerals are added and the duration of the incubation period corresponds to the amount of minerals that are metabolized by the microalgae. When the supplemental minerals are added to the microalgae after harvest from the growing vessel and are no longer in growth phase, the microalgae have less time to metabolize the minerals which results in more binding of the minerals to the microalgae cell walls. The amount of minerals that are bound and reversibly chelated, instead of metabolized may be also related to the proportion or concentration at which the supplemental minerals are added to the microalgae, and other factors such as temperature and pH.

In some embodiments, the supplemental minerals may be added at a specific concentration. In some embodiments, the supplemental minerals may be added in a specific proportion to the amount of microalgal biomass. In some embodiments, the supplemental minerals may be added to the microalgae culture when the microalgae culture is at a specific pH. In some embodiments, the supplemental minerals may be added to a specific mass of microalgae. In some embodiments, the supplemental minerals may be given a specific time duration in which to bind to the microalgae by assimilation, reversible chelation, and/or absorption. Reversible chelation may refer to the ionic binding of minerals to a cell wall and or exo-polysaccharides of the microalgae. Reversible chelation may not require metabolization of the mineral and may be based on an ion exchange process. The process may be reversible and therefore may allow the mineral ion to release in the conditions of a pH change, such as it a digestive track.

In some embodiments, the supplemental minerals may be added after the microalgae culture is concentrated to a certain concentration of solids by dewatering or other known methods of concentrating a microalgae culture. In some embodiments, the supplemental minerals may be added before the microalgae culture is concentrated to a certain concentration of solids by dewatering or other known methods of concentrating a microalgae culture. In some embodiments, the enriched microalgae may be dewatered to a specific concentration of solids. In further embodiments, the dewatered microalgae may comprise a wet solution. In further embodiments, the dewatered .microalgae may comprise a microalgal paste. In various embodiments of the present invention, “algal paste” and/or “microalgal paste” may refer to a partially dewatered algal or microalgal culture having fluid properties that allow it to flow. Generally an algal of microalgal paste may have a water content of about 90%.

In various embodiments, the dewatered microalgae may comprise a microalgal cake. An “algal cake” and/or “microalgal cake” may refer to a partially dewatered algal or microalgal culture that lacks the fluid properties of an algal of microalgal paste and/or tends to clump. Generally an algal or microalgal cake may have a water content of about 60% or less.

In one embodiment, the dewatered microalgae may comprise a free flowing powder. In some embodiments, the dewatered microalgae may be stabilized by methods such as, but not limited to, drying, cooling, freeze drying and freezing.

In one non-limiting example of the above method, the microalgae may be harvested from a growing vessel by centrifugation concentrating the microalgae at levels up to 50-200 g Dry Weight (DW)/liter to produce a microalgal paste. The resulting harvested microalgal paste has supplemental minerals comprising one or more mineral salt (containing minerals such as Se, Fe, Mn, Zn, and Cu) added to the microalgal paste at a concentration less than 15-0.1 g/liter, and is then incubated in an enrichment medium for 15-120 minutes. The incubation is carried out at a temperature of 5-40 degrees C., a pH of 6-12, and orbital shaking at 25-150 rpm. For further enrichment of the microalgae, the supplemental minerals comprising one or more mineral salts could be added to the culture medium 1-2 days before the microalgae are harvested. The incubated microalgal paste enriched with minerals is batch centrifuged, with the resulting supernatant being recycled back to the enrichment medium. The resulting enriched microalgal solids are stabilized by known methods such as, but not limited to, freezing, refrigerated storage, freeze drying, spray drying, or drum drying to produce an enriched microalgae product.

In some embodiments, the method further comprises the step of feeding the enriched microalgae product directly to any suitable aquatic animal such as an adult fish. The microalgae product may also be directly fed to other aquatic animals such as, for example, oysters, mollusks, scallops, and/or shrimp. In some embodiments, the method further comprises the step of mixing the enriched microalgae product in an aquafeed comprising additional ingredients. In some embodiments, the method further comprises mixing the microalgae in an aqua feed to comprise a specific percent of the aquafeed. In some embodiments, the additional aquafeed ingredients comprise fishmeal or fish oil.

With the above method, which adds supplemental minerals directly to natural microalgae and feeds the enriched microalgae product directly to adult fish/aquatic animals or using the enriched microalgae product directly in an aquafeed mixture, the prior art steps of: encapsulating a preparation, feeding the microalgae to another live feed (e.g. rotifers) before consumption by fish, and genetic modification of the microalgae are not required. Eliminating these steps increases the efficiency of the process of making an aquafeed, and reduces the costs associated with the time and resources required to: encapsulate a preparation; grow, maintain, and handle an additional live feed; and genetically modify a microalgal strain.

The method described above produces an enriched microalgae product for use as an aquafeed for aquatic animals (e.g. adult fish, oysters, mollusks, scallops, and shrimp). The various parameters of the method may be adjusted to produce an aquafeed of a desired composition and mineral profile matching the nutritional requirements of a specific fish or aquatic animal.

In some embodiments, the resulting enriched microalgae product may be combined in an aquafeed composition comprising a percentage of the enriched microalgae. The level of inclusion in the aquafeed depends on the fish nutritional requirements for the mineral(s) of interest and a mineral's bioaccumulation capacity with the species of microalgae used in the above described method. In some embodiments, the enriched microalgae product comprises less than 1% of an aquafeed for adult fish. In some embodiments the enriched microalgae product comprises about 1% of an aquafeed for adult fish. For example, in one embodiment, the microalgae product may comprise less than 1% of microalgae enriched with a profile of assimilated, reversibly chelated, and absorbed minerals specific to the nutritional requirements of an adult fish; and a remainder comprising at least one or more other ingredients from the group consisting of fishmeal and fish oil. In another embodiment, the microalgae product may be an animal feed product comprising at least 0.1% to about 1-5% of microalgae enriched with a profile of assimilated, reversibly chelated, and/or absorbed minerals specific to the nutritional requirements of the animal.

While the percent inclusion of enriched microalgae for the application of providing supplemental minerals in an aquafeed is small, such as at least 0.1%, and in some embodiments about 1% or less, using the enriched microalgae in a different application in an aqua feed will change the percent of inclusion. In some embodiments, the microalgae are used in dietary applications such as, but not limited to, probiotics, protein supplementation, amino acid supplementation, fatty acid supplementation, vitamin supplementation, and carbohydrate supplementation; and comprise about 5% or less of an aquafeed. In further embodiments, the enriched microalgae comprise about 5-10% of an aquafeed. In some embodiments, the enriched microalgae are used to entirely replace fishmeal and comprise about 50-80% of an aquafeed. In further embodiments, the enriched algae comprise about 70% of an aquafeed.

Several experiments were performed to illustrate various exemplary embodiments of methods for enriching microalgae with essential minerals and/or to illustrate the concept of mineral delivery using microalgae as a vehicle.

### EXAMPLE 1

Nannochloropsis is cultured following standard procedures known in the art and harvested directly from an aqueous culture medium by centrifugation, without the use of any flocculants. The thy weight of the harvested microalgae is adjusted to 50 g/liter through the addition of a salt water medium. Using the harvested microalgae, an experiment is run in triplicate using a 250 ml shake flask with 100 ml running volume. Salt comprising trace minerals (Zn, Se, Mn, Cu or Fe) is added to the harvested microalgae at around 1 g/liter, depending on the type of inorganic mineral. The flasks are incubated at 50 g CDW (cell dry weight) Nannochloropsis microalgae per liter, a temperature of 30 degrees C. a pH of 8, and 120 rpm for time periods of 0, 15, 30, 60, 120 and 180 minutes. The initial pH of the medium is set with NaOH (1M) and H2SO4 (1M). At each sampling point (0, 15, 30, 60, 120, and 180 minutes) a 20 ml sample is taken and centrifuged (3000 g, 30 degrees C., 5 minutes). with the time 0 minutes sample being taken right before the addition of the salt comprising trace minerals. The resulting pellet is freeze dried and the supernatant is frozen. The mineral analysis of the resulting pellet and supernatant is made by atomic adsorption spectrophotometry (AOAC 0968.008 and AOAC 0996.16). The results show the mineral content of the Nannochloropsis samples achieved with the different incubation time periods, which enables a determination of the optimal incubation time period for enriching Nannochloropsis with the identified minerals.

### EXAMPLE 2

Nannochloropsis is enriched according to the method developed in Example 1 and stabilized by freeze drying. Using the enriched microalgae, an experiment is run where the freeze dried microalgae biomass is re-suspended in fresh water to achieve 50 g CDW/liter. The suspension is mixed with a kitchen blender for 2 minutes to ensure the release of the single microalgae cells to the medium. 100 ml of the suspension is poured into a 250 ml Erlenmeyer flask and placed in an orbital incubator at a temperature of 30 degrees C. and 180 rpm. The initial pH of the suspension is 8, and the pH is decreased in a stepwise manner to pH levels of 7, 6, 5, 4, 3 and 2. The decrease in pH is achieved through the addition of 1M solution of NaOH. After 5 minutes of incubation at each pH level, a 20 ml sample of the suspension from each pH set point (8, 7, 6, 5, 4, 3, and 2) is centrifuge (3000 g, 30 degrees C., 5 minutes). The resulting pellet and supernatant is freeze dried before performing mineral analysis with atomic adsorption spectrophotometry (AOAC 0968.08 and AOAC 0996.16). The results show the amount of minerals released at each pH level by the enriched microalgae, enabling a determination of the amount of minerals that will be released by the enriched microalgae at pH levels achieved within the digestive systems of animals fed the enriched algae.

### EXAMPLE 3

Nannochloropsis is enriched with a blend of minerals according to the method developed in Example 1. In this experiment, the blend of minerals added to the microalgae matches the ratios of the nutritional requirements of adult Atlantic salmon. After the incubation period, the microalgae biomass is centrifuged. The resulting pellet and supernatant are analyzed to determine the mineral profile of the microalgae following the chelation process. The mineral profile of the microalgae is then compared to the nutritional requirements of the Atlantic salmon to determine if the ratios of enrichment are preserved during the chelation process. Based on the results of the mineral analysis, the interaction between the minerals during the chelation process is determined. The experiment is then repeated with a blend of minerals adjusted to account for the interactions between the minerals during the chelation process to achieve the nutritional requirements of adult salmon.

### EXAMPLE 4

The mineral enriched microalgal biomass produced according to Example 1 and Example 3 is used to manufacture commercial Atlantic Salmon pellets according to commercial extrusion process. The diets contain 0.5% microalgae biomass in dry weight to which the minerals are attached. The microalgae ingredient utilizing dietary enrichment with inorganic mineral salts is used to produce the diet of reference. This diet includes 0.2% of mineral salts on their composition, at least ten times more minerals than the microalgae based diets.

The experimental diets were fed to Juvenile Atlantic salmons for three. consecutive months in triplicate tanks. The fish grew in 1000 liter tanks with a stocking density of 4 kg/m³ and 12 h light photoperiod. The juveniles were fed “add libitum” twice a day recirculation of 10% volume/day. At the beginning, middle, and end of the experiment, each tank was sampled for standard length and body weight gain of the salmon. Blood and muscle samples were taken at the end of the experiment and mineral content on the muscle and blood samples were analyzed by atomic adsorption spectrophotometry (AOAC 0968.08 and AOAC 0996.16). Salmon feces, the pellets deposited in the pond, the fresh water, and spent water of the tank were collected to analyze the leaching of minerals into the water medium.

Results showed a similar growth pattern and body mineral content between the mineral enrichment protocols used in the diet, demonstrating the capacity of microalgae to deliver minerals in a water body more efficiently. The experiment demonstrated that the extra minerals used to formulate the diet, containing inorganic mineral, were lost through the leaching into the water body and through the defecation. The process of utilizing microalgae as a mineral enrichment method proved to be more efficient with regards to the overall amount of minerals used and also in terms of maintaining the water quality.

While the various embodiments discussed above for the enriched microalgae may be a mineral supplement in an aquafeed for adult fish, the enriched microalgae may also be a vehicle to provide a tailored mineral profile having a variety of applications. As described above, the microalgae may be enriched to produce a variety of nutritional profiles based on the types of minerals added, the concentration of minerals added, the species of microalgae uptaking the minerals, the timing of adding the minerals for uptake by the microalgae, and other factors which may affect the mineral, protein, amino acid, fatty acid, vitamin, or carbohydrate profiles of the microalgae. Using the above method, the nutritional profile of the microalgae may be tailored for the nutritional requirements of any aquatic and or non-aquatic animals, and used in a nutritional feed for such animals.

In some embodiments, the nutritional profile of the enriched microalgae may be customized for the nutritional requirements of non-aquatic animals such as livestock (e.g. cattle and other bovine, swine, chickens, turkeys, goats, bison, sheep, and water buffalo), equine (e.g. horse, donkeys, mules, and zebras), ungulates (e.g. horse, zebra, donkey, cattle/bison, rhinoceros, camel, hippopotamus, tapir, goat, pig, sheep, giraffe, okapi, moose, elk, deer, antelope, and gazelle), pets (e.g. dogs, cats, rabbits, and guinea pigs), poultry (e.g. chickens, turkeys, ducks, geese, and ostriches), game animals (e.g. pheasants and quails), exotic/zoo animals (e.g. non-human primates) and other domesticated animals. The enriched microalgae may be used in various forms (e.g. aqueous solution, paste, cake, powder, flakes, and pellets) within a feed for such animals. The animal feed may comprise a mixed product comprising a certain percent of enriched microalgae with the remainder comprising other ingredients.

The nutritional requirements for aquatic and non-aquatic animals, comprising protein, amino acids, fatty acids, vitamins, carbohydrates, macro minerals, and trace mineral requirements may be obtained from a variety of published sources. Such publications and sources of publications on animal nutritional requirements include, but are not limited to, the Merck Veterinary Manual; reports published by the National Research Council (NRC) of the National Academies; and papers, conference presentations and webpages published by educational institutions, cooperatives, and extension systems (e.g. North Dakota State University, Alabama Cooperative Extension System, University of Tennessee, and Mississippi State University Extension Service). For example, according to the NRC report on the Nutritional Requirements of Dogs and Cats, the daily recommended allowance of minerals for an adult dog weighing 33 pounds and consuming 1,000 calories per day comprises: 0.75 g Calcium, 0.75 g Phosphorus, 150 mg Magnesium, 100 mg Sodium, 1 g Potassium, 150 mg Chlorine, 7.5 mg Iron, 1.5 mg Copper, 15 mg Zinc, 1.2 mg Manganese, 90 μg Selenium, and 220 μg Iodine. An example of the nutritional requirements for a gestating beef cow (in mg mineral per kg dry diet) provided by the NRC report on the Nutritional Requirements of Beef Cattle comprises: 0.10 mg/kg Cobalt, 10 mg/kg Copper, 0.50 mg/kg Iodine, 50 mg/kg Iron, 40 mg/kg Manganese, 0.10 mg/kg Selenium, and 30 mg/kg Zinc. These published nutritional requirements can be used with the above described system to produce enriched microalgae products for feeding different animals.

The above method may also be used to produce a fertilizer composition and/or phyto-nutrient product comprising the microalgae product enriched with minerals for the nutritional profiles of plants. In one embodiment, the fertilizer composition may be configured to be a liquid, a dry flake, and/or a powder. The essential nutrients for plants may include primary nutrients, secondary nutrients, and micronutrients capable of being assimilated, reversibly chelated, and absorbed by microalgae. The primary nutrients that may be enriched into the microalgae product include Nitrogen (N), Phosphorus (P), and Potassium (K). The secondary nutrients that may be enriched into the microalgae product include Sulfur (S), Calcium (Ca), and Magnesium (Mg). The micronutrients that may be enriched into the microalgae product include Zinc (Zn), Iron (Fe), Copper (Cu), Manganese (Mn), Boron (B), Molybdenum (Mo), and Chlorine (Cl). In various embodiments of the present invention, the microalgae product may be enriched with the primary nutrients, secondary nutrients, and/or the micronutrients in a non-metabolized form. In addition to the mineral delivery capability of microalgae, other nutrients such as lipids, amino acids and vitamins can be provided to plants, crops and/or soil by microalgae. In some embodiments, the enriched microalgae may be used as a fertilizer or an ingredient of a fertilizer for plants, crops and/or soil. In further embodiments, the fertilizer is distributed to plants, crops and/or soil with water through irrigation systems such as, but not limited to, drip lines or spraying. Spraying applications may comprise spraying a solution directly on the plant leaves, plant stems, plant stalks, plant vines, the airspace immediately proximate to the plant, and/or the ground immediately proximate to the plant. In further embodiments, the fertilizer may be distributed to plants, crops and/or soil in a dry flake or powder form. Dry flake or powder applications may comprise shaking or sprinkling directly on the leaves, stalk or vine; shaking or sprinkling directly on the ground immediately proximate to the plant; and/or mixing the flakes or powder with the soil in which the plant is growing or will be planted.

In some embodiments, the enriched microalgae transfer nutrients from the microalgae cell to the plant cells in the leaf system through cytoplasmic streaming. In some embodiments, the enriched microalgae transfer nutrients from the microalgae cell to the plant cells in the root system through cytoplasmic streaming. In further embodiments, the nutrients not transferred from the microalgae cell to the plant cells in the root system through cytoplasmic streaming are released into the soil.

The amount of fertilizer or phyto-nutrient product to use and methods of applying fertilizer and phyto-nutrient products vary based on the condition of the soil, time of year, plant yield, and the type of plant growing in the soil. Recommendations are provided by government entities such as, but not limited to, state university extension systems (e.g. Washington State Extension Programs), local agriculture divisions (e.g. Government of Alberta Agriculture and Rural Development), and the Food and Agriculture Organization (FAO) of the United Nations. For example, the Alberta Agriculture and Rural Development's recommendation for sufficient nutritional requirements of spring wheat in growth stage include: 2.0-3.0% N, 0.26-0.5% P, 1.5-3.0% K, 0.1-0.15% S, 0.1-0.2 Ca, 0.1-0.15% Mg, 10-15 ppm Zn, 3.0-4.5 ppm Cu, 15-20 ppm Fe, 10-15 ppm Mn, 3-5 ppm B and 0.01-0.02 ppm Mo in the whole plant prior to filling. The microalgae mineral profile may also be customized for the nutritional requirements of house plants (e.g. ferns), flowers, agricultural crops (e.g., wheat, corn, grain sorghum, soybeans, canola, milo, barley, sugarcane, pumpkins, rice, cassava, tobacco, hay, potatoes, cotton, beets, strawberries), fruit trees and bushes (e.g., apple, orange, grapefruit, lemon, lime, raspberries, blackberries), nut trees and bushes (e.g. pecan, butternut, walnut, almond, chestnut), fruit vines (e.g. grapes, melons, kiwi), grasses, and residential landscaping plants.

One demonstration of the capability of enriched microalgae to deliver nutrients to plants may be provided by the use of enriched Chlorella vulgaris. When additional Phosphorus is added to the culture medium, Chlorella vulgaris is known to be able to assimilate and store between 1.7 and 3.5 times more Phosphorus than the Chlorella vulgaris requires. The enriched Chlorella can be administered to plants as a fertilizer or as an ingredient of a fertilizer through a drip line or spray application and supply significant amounts of Phosphorus in a water soluble form, as well as numerous other proteins, amino acids, and micronutrients contained in the microalgae.

In another embodiment, the mineral enriched microalgae may be combined in a solution with herbicides and pesticides that are applied to plants, crops, and/or the soil. The combination with herbicides and pesticides allows the nutrients to be supplied to the plants, crops, and/or soil in a single application with pest and weed control benefits.

Several experiments are run to optimize the method of fertilizing plants with mineral enriched microalgae, and to optimize the method of enriching microalgae with the nutritional profile specific to a plant.

### EXAMPLE 5

The goal of this experiment is to determine the volume of mineral enriched microalgae fertilizer at which the plant stops uptaking nutrients and the minerals are lost to the soil. Chlorella is enriched With a blend of minerals, including Phosphorus, according to the methods disclosed above. In this experiment, the blend of minerals added to the microalgae matches the ratios of the nutritional requirements of a plant. After the incubation period, a fertilizer solution comprising enriched Chlorella and water, with a determined concentration of solids (enriched microalgae), is applied to soil in a series of paired containers. Each pair of containers comprises one container with the contents comprising soil only, and one container with the contents comprising soil and the plant. All other container inputs such as light, air, etc., are identical for each container and held constant. Different volumes of the fertilizer solution are administered to each container pair through a drip irrigation system, with each volume of fertilizer solution having the same solids concentration. Soil samples from each container are taken before the application of the fertilizer solution, and at time intervals of 1 hour, 2 hours, 3 hours, 6 hours, 12 hours, and 24 hours after the application of the fertilizer solution. The soil samples are analyzed for their mineral composition. The mineral compositions of the soil samples are compared to determine the volume of fertilizer solution at which the nutrients of the fertilizer solution are no longer transferred to the plant or uptaken by the root system, and remain in the soil. From this experiment which varies the volume of enriched mineral fertilizer solution used, it is desired to learn the most efficient volume of fertilizer solution in which the delivery of minerals to the plant is maximized, and the loss of minerals and microalgae to the soil is minimized, therefore maximizing the cost effectiveness of the enriched microalgae fertilizer.

The experiment is then repeated using a fertilizer solution comprising water and inorganic minerals in place of the fertilizer solution enriched microalgae and water. The results of the soil sample analysis for both the enriched microalgae fertilizer solution experimental run and the inorganic mineral fertilizer solution experimental run are compared to determine the efficiency increase in delivery of minerals to the plant through the use of microalgae as a mineral vehicle as opposed to the use of inorganic minerals.

### EXAMPLE 6

The goal of this experiment is to determine the concentration of mineral enriched microalgae fertilizer at which the plant stops uptaking nutrients and the minerals are lost to the soil. Chlorella is enriched with a blend of minerals including Phosphorus according to the methods disclosed above. In this experiment, the blend of minerals added to the microalgae matches the ratios of the nutritional requirements of a plant. After the incubation period, a series of fertilizer solutions comprising enriched Chlorella and water at different concentrations of solids (enriched microalgae) are applied to soil in a series of paired containers. Each pair of containers comprises one container with the contents comprising soil only, and one container with the contents comprising soil and the plant. All other container inputs such as light, air, etc., are identical for each container and held constant. The same volume of the fertilizer solutions are added to each container pair through a drip irrigation system, which each volume of fertilizer solution having different solids concentrations. Soil samples from each container are taken before the application of the fertilizer solution, and at time intervals of 1 hour, 2 hours, 3 hours, 6 hours, 12 hours, and 24 hours after the application of the fertilizer solution. The soil samples are analyzed for their mineral composition. The mineral compositions of the soil samples are compared to determine the concentration of enriched algae at which the nutrients of the fertilizer solution are no longer transferred to the plant or uptaken by the root system, and remain in the soil. From this experiment which varies the concentration of enriched algae in the fertilizer solution, it is desired to learn the most efficient concentration of fertilizer solution in which the delivery of minerals to the plant is maximized, and the loss of minerals and microalgae to the soil is minimized, therefore maximizing the cost effectiveness of the enriched microalgae fertilizer.

The experiment is then repeated using a fertilizer solution comprising water and inorganic minerals in place of the fertilizer solution enriched microalgae and water. The results of the soil sample analysis for both the enriched microalgae fertilizer solution experimental fun and the inorganic mineral fertilizer solution experimental run are compared to determine the efficiency increase in delivery of minerals to the plant through the use of microalgae as a mineral vehicle as opposed to the use of inorganic minerals.

### EXAMPLE 7

The goal of this experiment is to supply a plant with the required nutritional profile using a mineral enriched strain of microalgae. Using the above disclosed method, Chlorella is enriched with a profile of minerals specific to the nutritional requirements of spring wheat in growth stage through the addition of a blend of nitrogen, phosphorus, potassium, sulphur,calcium, magnesium, zinc, copper, iron, manganese, boron, and molybdenum, to a culture of Chlorella. The nutritional profile specific to the wheat comprises 2.0-3.0% N, 0.26-0.5% P, 1.5-3.0% K, 0.1-0.15% S, 0.1-0.2% Ca, 0.1-0.15% Mg, 10-15 ppm Zn, 3.0-4.5 ppm Cu, 15-20 ppm Fe, 10-15 ppm Mn, 3-5 ppm B and 0.01-0.02 ppm Mo in the whole plant prior to filling. After the incubation period, the microalgae biomass is centrifuged. The resulting solids and supernatant are analyzed to determine the mineral profile of the microalgae following the chelation process. The mineral profile of the microalgae is then compared to the nutritional requirements of spring wheat in growth stage to determine if the ratios of enrichment are preserved dining the chelation process. Based on the results of the mineral analysis, the interaction between the minerals during the chelation process is determined. The experiment is then repeated with a blend of minerals adjusted to account for the interactions between the minerals during the chelation process to achieve the nutritional requirements of spring wheat in growth stage. The enriched Chlorella cells are administered to the wheat through a spray or drip irrigation system in a fertilizer solution comprising water.

In the foregoing description, the invention has been described with reference to specific exemplary embodiments. Various modifications and changes may be made, however, without departing from the scope of the present invention as set forth. The description and figures are to be regarded in an illustrative manner, rather than a restrictive one and all such modifications are intended to be included within the scope of the present invention. Accordingly, the scope of the invention should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present invention and are accordingly not limited to the specific configuration recited in the specific examples.

Benefits, other advantages and solutions to problems have been described above with regard to particular embodiments. Any benefit, advantage, solution to problems or any element that may cause any particular benefit, advantage or solution to occur or to become more pronounced, however, is not to be construed as a critical, required or essential feature or component.

The terms “comprises”, “comprising”, or any variation thereof, are intended to reference a non-exclusive inclusion, such that a process, method, article, composition, system, or apparatus that comprises a list of elements does not include only those elements recited, but may also include other elements not expressly listed or inherent to such process, method, article, composition, system, or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

The present invention has been described above with reference to an exemplary embodiment. However, changes and modifications may be made to the exemplary embodiment without departing from the scope of the present invention. These and other changes or modifications are intended to be included within the scope of the present invention.

What is claimed is:

1. A method of making a microalgae product enriched with non-metabolized minerals, comprising:
a. Growing a culture of microalgae in an aqueous culture medium;
b. Harvesting the microalgae by separating the microalgae from the aqueous culture medium;
c. Adding supplemental minerals specific to a profile of nutritional requirements for an animal to the microalgae;
d. Incubating the microalgae and the supplemental minerals to facilitate the microalgae assimilating, reversibly chelating, and absorbing the supplemental minerals to produce a microalgae product enriched with non-metabolized minerals specific to the profile of nutritional requirements of the animal.

2. The method of claim 1 further comprises the step of
a. Dewatering the microalgae product enriched with minerals to further reduce the water content of the microalgae product.

3. The method of claim 2 further comprising the step of
a. Stabilizing the microalgae product enriched with minerals.

4. The method of claim 1, wherein the supplemental minerals are added to the microalgae before harvesting the microalgae.

5. The method of claim 1, wherein the supplemental minerals are added to the microalgae after harvesting the microalgae.

6. The method of claim 1, wherein the animal is an aquatic animal selected from the group consisting of: adult fish, oysters, mollusks, scallops, and shrimp.

7. The method of claim 6, further comprising the step of mixing the microalgae with at least one from the group consisting of fishmeal and fish oil, to produce an aquafeed.

8. The method of claim 6, wherein the microalgae are fed directly to the aquatic animal.

9. The method of claim 7, wherein the aquafeed is fed directly to the aquatic animal.

10. The method of claim 1, wherein the supplemental minerals comprise at least one from the group consisting of: boron, bromine, calcium, chloride, chromium, cobalt, copper, iodine, iron, magnesium, manganese, molybdenum, nickel, phosphorus, potassium, selenium, sodium sulphur, vanadium and zinc.

11. The method of claim 1, wherein the microalgae is selected from at least one from the group consisting of: Nannochloropsis, Chlorella, Spirulina, Schizochytrium, Crypthecodinium, and Scenedesmus.

12. The method of claim 1, wherein the supplemental minerals are added at a mineral concentration of less than about 15 to 0.1 g/liter to the harvested microalgae of a concentration of about 50-200 g microalgae DW/liter.

13. The method of claim 1, wherein the supplemental minerals and microalgae are incubated for 15-120 minutes.

14. The method of claim 1, wherein the supplemental minerals and microalgae are incubated at a temperature of about 5-40 degrees C.

15. The method of claim 1, wherein the supplemental minerals and microalgae are incubated at a pH of about 6-12.

16. The method of claim 1, wherein the supplemental minerals and microalgae are incubated with orbital shaking at about 25-150 rpm.

17. The method of claim 1, wherein the supplemental minerals are added to the culture of microalgae 1-2 days before the harvesting of the microalgae.

18. The method of claim 3, wherein the Method of stabilizing is selected from the group consisting of: freezing, refrigeration, freeze drying, spray drying, or drum drying.

19. A mineral supplement composition for an aquatic animal the composition comprising a microalgae product enriched with at least one mineral from the group consisting of arsenic, chromium, cobalt, copper, fluorine, iron, iodine, lead, lithium, manganese, molybdenum, nickel, selenium, silicon, vanadium, zinc in a non-metabolized form.

20. The mineral supplement composition of claim 19, wherein the aquatic animal is selected from the group consisting of: adult fish, oysters, mollusks, scallops, and shrimp.

21. An aquafeed product for adult fish, comprising less than 1% of microalgae enriched with a profile of assimilated, reversibly chelated, and absorbed minerals specific to the nutritional requirements of an adult fish; and a remainder comprising at least one of more other ingredients from the group consisting of fishmeal and fish oil.

22. A mineral supplement composition for a non-aquatic animal, the composition comprising a microalgae product enriched with at least one mineral from the group consisting of boron, bromine, calcium, chloride, chromium, cobalt, copper, iodine, iron, magnesium, manganese, molybdenum, nickel, phosphorus, potassium, selenium, sodium, sulphur, vanadium and zinc in a non-metabolized form.

23. The mineral supplement composition of claim 22, wherein the non-aquatic animal is selected from the group consisting of: poultry, horses, ungulates, game animals, bovine, pets, pigs.

24. A fatty acid supplement composition for animals, comprising a microalgae product comprising at least one fatty acid of chain length between C10 and C24 and enriched with at least one mineral from the group consisting of: arsenic, boron, bromine, calcium, chloride, chromium, cobalt, copper, fluorine, iodine, iron, lithium, magnesium, manganese, molybdenum, nickel, phosphorus, potassium, selenium, silicon, sodium, sulphur, vanadium and zinc in a non-metabolized form.

25. An animal feed product, comprising at least 0.1% of microalgae enriched with a profile of assimilated, reversibly chelated, and absorbed minerals specific to the nutritional requirements of an animal.

26. The animal feed product of claim 25, wherein the product comprises about 1-5% of microalgae enriched with a profile of assimilated, reversibly chelated, and absorbed minerals specific to the nutritional requirements of an animal.

27. The animal feed product of claim 25, wherein the product comprises about 5-10% of microalgae enriched with a profile of assimilated, reversibly chelated, and absorbed minerals specific to the nutritional requirements of an animal.

28. The animal feed product of claim 25 wherein the product comprises about 50-80% of microalgae enriched with a profile of assimilated, reversibly chelated, and absorbed minerals specific to the nutritional requirements of an animal.

29. The animal feed product of claim 25, wherein the product comprises about 1% or less of microalgae enriched with a profile of assimilated, reversibly chelated, and absorbed mineral's specific to the nutritional requirements of an animal.

30. An aquafeed product for adult fish, comprising:
a. Microalgal biomass, wherein the microalgal biomass is a mineral enriched microalgae product providing a mineral profile comprising at least one from the group consisting of:
i. About 30-170 mg Iron per kg of dry aquafeed;
ii. About 1-5 mg Copper per kg of dry aquafeed;
iii. About 2-20 mg Manganese kg of thy aquafeed;
iv. About 15-40 mg Zinc per kg of dry aquafeed;
v. About 0.05-1.0 mg Cobalt per kg of dry aquafeed;
vi. About 0.15-0.5 mg Selenium per kg of thy aquafeed;
vii. About 1-4 mg Iodine per kg of thy aquafeed; and
b. At least one or more additional ingredients.

31. A dog food product for adult dogs, comprising:
a. Microalgal biomass, wherein the microalgal biomass is a mineral enriched microalgae product providing a mineral profile comprising at least one from the group consisting of:
i. About 0.75 g Calcium per 1000 calories of dog food;
ii. About 0.75 g Phosphorus per 1000 calories of dog food;
iii. About 150 mg Magnesium per 1000 calories of dog food;
iv. About 100 mg Sodium per 1000 calories of dog food;
v. About 1 g mg Potassium per 1000 calories of dog food;
vi. About 150 mg Chlorine per 1000 calories of dog food;
vii. About 7.5 mg Iron per 1000 calories of dog food;
viii. About 1.5 mg Copper per 1000 calories of dog food;
ix. About 15 mg Zinc per 1000 calories of dog food;
x. About 1.2 mg Manganese per 1000 calories of dog food;
xi. About 90 μg Selenium per 1000 calories of dog food;
xii. About 220 μg Iodine per 1000 calories of dog food; and
b. At least one or more additional ingredients.

32. A cattle feed product for gestating beef cows, comprising:
a. Microalgal biomass, wherein the microalgal biomass is a mineral enriched microalgae product providing a mineral profile comprising at least one from the group consisting of:
i. About 0.10 mg Cobalt per kg of dry cattle feed;
ii. About 10 mg Copper per kg of dry cattle feed;
iii. About 0.50 mg Iodine per kg of dry cattle feed;
iv. About 50 mg Iron per kg of dry cattle feed;
v. About 40 mg Manganese per kg of dry cattle feed;
vi. About 0.10 mg Selenium per kg of dry cattle feed;
vii. About 30 mg Zinc per kg of dry cattle feed; and
b. At least one or more additional ingredients.

33. A fertilizer composition for plants, the composition comprising a microalgae product enriched with at least one mineral from the group consisting of: nitrogen phosphorus, potassium, sulfur, calcium, magnesium, zinc, iron, copper, manganese, boron, molybdenum, and chlorine in a non-metabolized form.

34. The composition of claim 33, wherein the composition is a liquid.

35. The composition of claim 34, wherein the liquid composition is sprayed on at least one selected from the group consisting of: plant leaves, plant stalks, plant vines, the airspace immediately proximate to the plant, and the ground immediately proximate to the plant.

36. The composition of claim 33, wherein the composition is in the form of dry flakes or powder.

37. The composition of claim 36, wherein the dry flakes or powder are applied to at least one selected from the group consisting of: the ground immediately proximate to the plant, and soil in which a plant is growing or will be planted.

38. A method of making a microalgae product enriched with non-metabolized minerals, comprising:
a. Growing a culture of microalgae in a culturing vessel comprising an aqueous culture medium and at least one pair electrodes submerged in the aqueous culture medium,
i. The electrode comprised of an electrode material comprising at least one of a mineral specific to a nutritional profile of an animal;
b. Applying an electric current to the at least one pair of electrodes sufficient to cause the electrode material to leach into the aqueous culture medium;
c. Incubating the microalgae to facilitate the microalgae assimilating, reversibly chelating, and absorbing of the electrode material to produce a microalgae product enriched with non-metabolized minerals specific to the profile of nutritional requirements of the animal;
d. Harvesting the microalgae product enriched with non-metabolized minerals to separate the microalgae product from the aqueous culture medium.

39. The method of claim 38, wherein the electrical current is direct, alternating, or pulsed.

40. The method of claim 38, wherein minerals comprise at least one from the group consisting of: boron, bromine, calcium, chloride, chromium, cobalt, copper, iodine, iron, magnesium, manganese, molybdenum, nickel, phosphorus, potassium, selenium, sodium, sulphur, vanadium and zinc.
